(19) United States
(12) Patent Application Publication
(10) Pub. No.: US 2023/0157994 A1
(43) Pub. Date: May 25, 2023

(54) NEW COMPOSITIONS AND METHODS OF TREATING COVID-19 DISEASE

(71) Applicants: 4LIVING BIOTECH, Lille (FR); UNIVERSITE DE BORDEAUX, BORDEAUX (FR); CENTRE HOSPITALIER DE BORDEAUX, TALENCE (FR); INSTITUT NATIONAL DE LA SANTE ET DE LA RECHERCHE MEDICALE - INSERM, PARIS (FR); INSTITUT PASTEUR DE LILLE, LILLE (FR); CENTRE NATIONAL DE LA RECHERCHE SCIENTIFIQUE, Paris (FR); UNIVERSITÉ DE LILLE, LILLE (FR)

(72) Inventors: Patrick BERGER, BORDEAUX (FR); Isabelle DUPIN, TALENCE (FR); Renaud PREVEL, BORDEAUX (FR); Chloé JAMES, Pessac (FR); Pierre-Olivier GIRODET, BORDEAUX (FR); Keren BISMUTH, Paris (FR); Revital RATTENBACH, Paris (FR); Jérôme BRETON, Euralille (FR); Francois TROTTEIN, TOURCOING (FR); Valentin SENCIO, LILLE (FR)

(73) Assignees: 4LIVING BIOTECH, Lille (FR); UNIVERSITE DE BORDEAUX, BORDEAUX (FR); CENTRE HOSPITALIER DE BORDEAUX, TALENCE (FR); INSTITUT NATIONAL DE LA SANTE ET DE LA RECHERCHE MEDICALE - INSERM, PARIS (FR); INSTITUT PASTEUR DE LILLE, LILLE (FR); CENTRE NATIONAL DE LA RECHERCHE SCIENTIFIQUE, Paris (FR); UNIVERSITÉ DE LILLE, LILLE (FR)

(21) Appl. No.: 17/922,865

(22) PCT Filed: May 5, 2021
(86) PCT No.: PCT/EP2021/061919

§ 371 Date: Nov. 2, 2022

(30) Foreign Application Priority Data
May 7, 2020 (EP) 20173595.8

(51) Int. Cl. A61K 31/395; A61K 31/662; A61K 45/06; A61P 31/14

(52) U.S. Cl. CPC A61K 31/395; A61K 31/662; A61K 45/06; A61P 31/14

(57) ABSTRACT

A method of treating COVID-19 in a patient. The patient may be in moderate to advanced stages of COVID-19 infection. The method includes administering to the patient a therapeutically effective amount of at least one antagonist or inhibitor of chemokine receptor CXCR4. The at least one antagonist or inhibitor of chemokine receptor CXCR4 is distinct from hydroxychloroquine.

### FIELD OF INVENTION

The invention relates to novel compositions and methods for the treatment of COVID-19 disease. More specifically, the invention relates to the use of at least one antagonist or inhibitor of chemokine receptor CXCR4 for use in the treatment of COVID-19 particularly in patients at moderate to severe stages of the disease.

### BACKGROUND OF THE INVENTION

Coronaviruses are known to cause severe respiratory and gastrointestinal diseases in animals The infection of humans with coronavirus strains have been described for many years to be associated with respiratory tract infections, i.e., common cold-like diseases. For example, SARS-CoV (Severe Acute Respiratory Syndrome-Corona Virus) is a highly aggressive human agent, causing acute respiratory distress syndrome (ARDS), with often fatal outcome. This virus appeared as an epidemic in 2003 after having crossed the species barriers from bats to civet cats and humans demonstrating the potential of coronaviruses to cause high morbidity and mortality in humans. The strains HCoV-NL63 and HCoV-HKU1 were discovered in 2004 and 2005, respectively.

On 31 Dec. 2019, the World Health Organization (WHO) China Country Office was informed of cases of pneumonia of unknown etiology detected in Wuhan City, Hubei Province of China. From 31 Dec. 2019 through 3 Jan. 2020, a total of 44 case-patients with pneumonia of unknown etiology were reported to WHO by the national authorities in China. During this reported period, the causal agent was not identified. By 1 Mar. 2020, 44 672 patients in China were reported with confirmed infection, 2.1% being below the age of 20. The most commonly reported symptoms included fever, dry cough, and shortness of breath, and most patients (80%) experienced mild illness. Approximately 14% experienced severe disease and 5% were critically ill. Early reports suggested that illness severity is associated with age (>60 years old) and co-morbid diseases. As on Apr. 29, 2020 there were 3,152,556 confirmed cases worldwide, with 218,491 reported dead due to COVID-19.

Severe acute respiratory syndrome-coronavirus 2 (SARS-CoV-2) has been identified as the emerging virus responsible for the pandemic COVID-19 now declared as a “global threat for public health” by the WHO. In most cases, clinical features of COVID-19 remain benign. However, COVID-19 condition can be much more severe and may require for oxygen support at hospital in 16 to 20% of patients. It can cause acute respiratory distress syndrome (ARDS) in 5 to 10% of patients with admission to intensive care unit (ICU) and invasive mechanical ventilation. Long ICU length of stay participates in healthcare resources overwhelming. ARDS is responsible for 99% of admission to ICU and COVID-19 related deaths. ARDS is characterized by a respiratory worsening triggered by gas exchange impairment secondary to alveolar-capillary barrier oedema. This lung injury results from a massive dysregulated inflammatory response to lung epithelial invasion by the virus.

SARS-Cov-2 has infected hundreds of millions of people since 2019 and represent a global health emergency requiring the development of potent antiviral treatments.

Despite continued research antiviral drugs against most hazardous and dangerous viral infections have not been developed, and the existing medicaments are often toxic to humans or insufficiently effective. Sor far only vaccines have been developed to prevent these pandemics. Most existing or under-development drugs act through a specific interaction with certain viral proteins, and such drugs have a limited spectrum of activity and promote a rapid emergence of resistant virus variants.

Therefore, it is an object of the present disclosure to provide new compositions and methods for the treatment of COVID-19 caused by SARS-Cov-2 which are expected to be especially effective in improving survival rate in moderate to severe cases. Surprisingly, it has been found that therapeutically effective amount of at least one antagonist or inhibitor of chemokine receptor CXCR4 provides critical support and health improvement in COVID-19 infected patients.

### SUMMARY OF THE INVENTION

The present application relates to a composition for use in a method of treating and to a method of treating respiratory viral disease caused by SARS-Cov-2 virus such as in particular COVID-19 comprising administering to a patient a therapeutically effective amount of at least one antagonist or inhibitor of chemokine receptor CXCR4, said antagonist or inhibitor of chemokine receptor CXCR4 being distinct from hydroxychloroquine.

Compositions and methods of treatment according to the present invention are particularly effective in improving survival in patients suffering from acute respiratory distress syndrome (ARDS) occurring in moderate to severe cases of respiratory viral infections caused by SARS-Cov-2 virus. Said compositions and methods of treatment are thus useful in for treating moderate to severe cases of COVID-19, and/or in cases of co-morbidity or multiple co-morbidities.

### BRIEF DESCRIPTION OF THE FIGURES

FIG. 1: is a graph showing murine lung function measured by the FEV0.05/FVC after daily subcutaneous injections of 1 mg/kg plerixafor during the 5 last weeks of the protocol. Mice tested presented lung injury induced by cigarette smoke exposure with poly-IC instillations, and either PBS injections (gray squares) or 1 mg/kg plerixafor injections (black squares). *: P<0.05.

FIGS. 2A-B: are graphs showing cardiac remodeling, quantified by Fulton index, calculated as RV/(LV+S), with LV: left ventricle, RV: right ventricle, S: septum. (A) Fulton index. Control mice (black circles) and mice with lung injury induced by cigarette smoke exposure, with poly-IC instillations (gray squares). (B) Fulton index. Tested mice presented lung injury induced by cigarette smoke exposure with poly-IC instillations, and either PBS injection (gray squares) or 1 mg/kg plerixafor injection (black squares). *: P<0.05, **: P<0.01.

FIGS. 3A-B: are graphs showing the effect of plerixafor on K18-hACE2 mice infected with SARS-CoV-2. (A) shows the individual percentage of body weight loss of vehicle-treated and plerixafor-treated mice on day 4 and 5 post infection with SARS-CoV-2. (B) shows the percentage of vehicle-treated and plerixafor-treated mice survival after infection with SARS-CoV-2.

FIG. 4: is a graph showing the quantification of Citrullinated histone H3 level in plasma of non-infected mice, infected mice with or without treatment with plerixafor (3 mg/kg/day). Mean±SEM; n=5 (or 2 for non-infected vehicle group).

FIGS. 5A-C: are graphs showing the effect of 10% COVID-19 sera from severe patients on neutrophils' migration. Neutrophils were isolated from patients with stable COPD (n=6) and were incubated with (black square symbol) or without (black circle symbol) 10% sera from patients with severe COVID-19. FIGS. 5A-C show comparison of (A) DAPI⁻CD16⁺, (B) DAPI⁻CD16⁺CXCR4⁻, and (C) DAPI⁻CD16⁺CXCR4⁺ cell migration in response to sera from patients with severe COVID-19. Results are expressed with symbols indicating individual subject values. * P<0.05, Wilcoxon matched pair test.

FIGS. 6A-C: are graphs showing the effect of plerixafor on migration of neutrophils isolated from patients with stable COPD (n=6) in response to 10% sera obtained from patients with severe COVID-19 in the presence of 25 μg/mL plerixafor (black square symbol) or vehicle (black circle symbol). FIGS. 6A-C show comparison of (A) DAPI⁻CD16⁺, (B) DAPI⁻CD16⁺CXCR4⁻, and (C) DAPI⁻CD16⁺CXCR4⁺ cell migration in response to sera from patients with severe COVID-19. Results are expressed with symbols indicating individual subject values. * P<0.05, Wilcoxon matched pair test.

### DETAILED DESCRIPTION

The present invention thus provides compounds, compositions, pharmaceutical compositions and methods of use of certain compounds that are antagonists or inhibitors of chemokine receptor CXCR4, for treating coronavirus infections caused by SARS-CoV2. Antagonists or inhibitors of chemokine receptor CXCR4 according to the present invention are distinct from hydroxychloroquine.

The present invention also relates to compounds, compositions, pharmaceutical compositions and methods of use of certain compounds that are antagonists or inhibitors of chemokine receptor CXCR4, for treating COVID-19, comprising administering to a patient a therapeutically effective amount at least one antagonist or inhibitor of chemokine receptor CXCR4. The present invention specifically provides a composition for use in a method of treating and a method of treating COVID-19 with or without multiple comorbidities comprising administering to said patient a therapeutically effective amount at least one antagonist or inhibitor of chemokine receptor CXCR4.

The present invention further relates to compounds, compositions, pharmaceutical compositions and methods of decrease the NETosis and/or mitigating pneumonia, acute respiratory failure and ARDS in SARS-Cov-2 infected patients comprising administering to said patient a therapeutically effective amount at least one antagonist or inhibitor of chemokine receptor CXCR4, with the proviso that said antagonists or inhibitors of chemokine receptor CXCR4 are preferably distinct from hydroxychloroquine.

As used herein, the term “treatment” or “treat” refer to curative or disease modifying treatment, including treatment of patient at risk of contracting the disease or suspected to have contracted the disease as well as patients who are ill or have been diagnosed as suffering from a disease or medical condition and includes suppression of clinical relapse. The treatment may be administered to a subject having a medical disorder or who ultimately may acquire the disorder, in order to cure, delay the onset of, reduce the severity of, or ameliorate one or more symptoms of a disorder or recurring disorder, or in order to prolong the survival of a subject beyond that expected in the absence of such treatment.

Acute respiratory failure or severe acute respiratory syndrome (SARS) may affect persons of all ages, with a higher likelihood for the first responders, e.g., nurses, physicians, etc. . . . . Initial symptoms include fever, chills, myalgia, cough, but also shortness of breath and/or tachypnea. Two third of the SARS-Cov-2 infected patients however developed complications such as ARDS.

According to the present invention, antagonist or inhibitor of chemokine receptor CXCR4 may be chosen among indole-based compound, a N-substituted indole compound, a bicyclam compound, a cyclam mimetic compound, a para-xylyl-enediamine-based compound, a guanidine-based antagonist compound, a tetrahydroquinolines-based compound, or a 1,4-phenylenebis(methylene) compound. Said antagonists or inhibitors of chemokine receptor CXCR4 according to the present invention are distinct from hydroxychloroquine and/or do not include hydroxychloroquine.

Preferred antagonist or inhibitor of chemokine receptor CXCR4 is chosen among plerixafor (AMD3100), Burixafor (TG-0054), JM1657, AMD3329, AMD3465, AMD070, MSX-122, CTCE-9908, WZ811, or BKT-140. Most preferred antagonist or inhibitor of chemokine receptor CXCR4 according to the present invention is plerixafor.

Plerixafor is well known drug and a CXCR4 antagonist, which has originally been developed as an anti-HIV drug. Also, the combination plerixafor and granulocyte-colony stimulating factor (G-CSF) was approved in 2008, by the US Food and Drug Administration to mobilize hematopoietic stem cells (HSCs) to the peripheral blood for collection and subsequent autologous transplantation in patients with non-Hodgkin's lymphoma and multiple myeloma. Plerixafor has been validated thru several clinical trials. Toxicology, pharmacokinetics as well as any potential adverse events are thus fully known, thereby allowing possible fast track of the registration for COVID-19 condition.

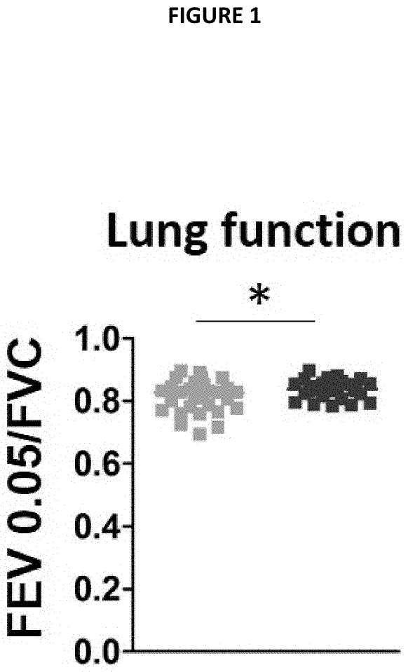

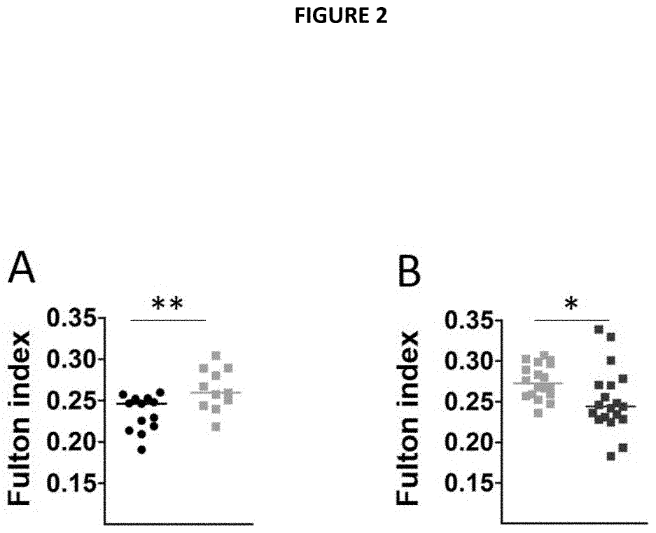

Applicants have showed in the Examples below that the administration of a therapeutically effective amount of CXCR4 antagonist, such as for example plerixafor (1 mg/kg) during the 5 weeks improved lung function of mice having lung injury induced by cigarette smoke exposure with poly-IC instillations, which mimic viral infection, and either PBS injection (FIGS. 1 and 2).

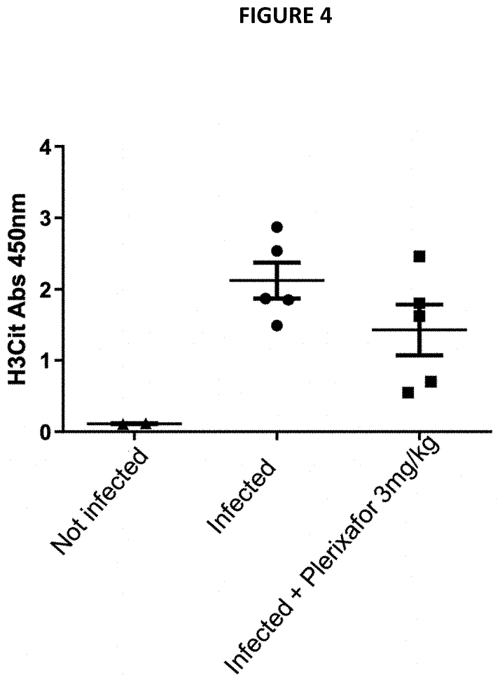

Applicants also showed that plerixafor may significantly improve respiratory conditions, and lung disease evolution of COVID-19 in patients at moderate or severe stages. In particular, it was showed that NETosis was significantly reduced in RNA virus infected mice after treatment with an effective amount of CXCR4 antagonist such as plerixafor (FIG. 4). These are clearly breakthrough results. Indeed, while the release of neutrophil extracellular traps (NETs) by neutrophils is known to be a physiological defense mechanism against invading microbial pathogens, the neutrophils and NETs have been also identified as contributing to the development of pathologic inflammation and ARDS secondary to respiratory viral infections, such as COVID-19. Indeed, NETosis, i.e., the formation of NETs in the alveolar space, has been local inflammation and viral burden to the lungs, causing acute respiratory distress syndrome (ARDS) in infected patients. Administration of a CXCR4 antagonist in patients is expected to result in significant inhibition of migration of neutrophils in COVID-19 patients.

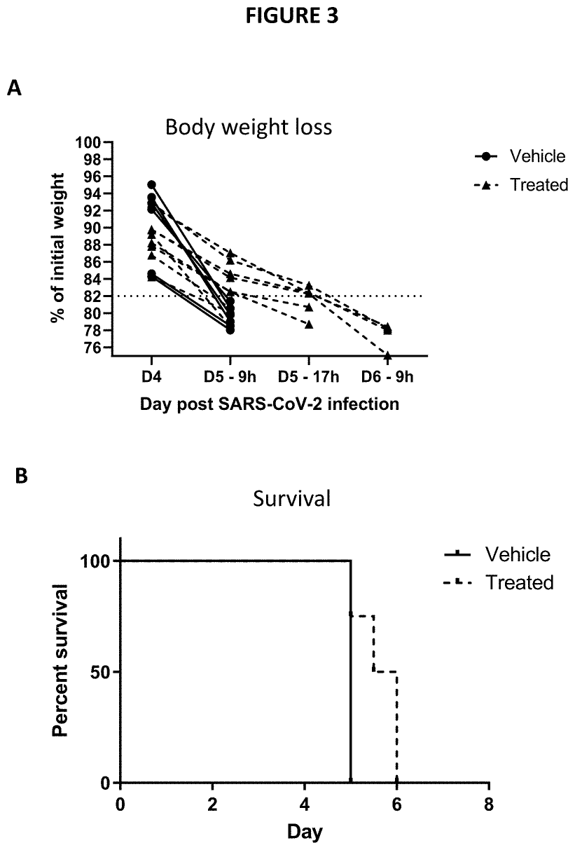

Furthermore, Applicants showed in a mouse model of severe COVID-19, using the K18-hACE2 transgenic mouse and a lethal dose of SARS-CoV-2 to mimic a severe COVID-19, that plerixafor reduced the mice body weight loss kinetic compared to vehicle-treated (FIG. 3A) and improved survival by 20% in 44,44% of plerixafor-treated mice (FIG. 3B).

This is particularly surprising since plerixafor has been disqualified in Feb. 24, 2020 article published by Hosseini F S et al. on the platform preprints.org (doi:10.20944/preprints202002.0438.v1). In this article, the authors performed a virtual screen to identify FDA-approved small molecules that present an inhibitory potential of the SARS-CoV-2 protease, based on a simulation of molecular docking and predicted binding energy within the binding pocket of the virus' protease. The authors expressly rejected clinical exploration of plerixafor for patients with COVID-19 given plerixafor known side effect of bone marrow and strong immune system suppression which was expected to lead to a worsening of the condition of the patients.

Compositions and methods according to the present invention are particularly efficient for treating COVID-19 patients at moderate to advanced or severe stage of the disease.

Moderate COVID-19 cases are those with inflammation lower down in the lungs, wherein lung symptoms like cough are more marked. The lungs consist of large airways (bronchi), smaller airways (bronchioles) and the tiny air sacs on the end (alveoli) where oxygen is extracted from the air. They contain a fluid called surfactant which keeps the lungs stretchy and compliant and helps keep the air sacs open. Patients with moderate COVID-19 may have inflammation moving down into the bronchioles. They are more breathless and tend to have an increased heart rate, particularly if they are moving around.

While the majority of patients with COVID-19 have benign or mild or moderate illness, 16 to 20% of affected patients develop severe COVID-19 conditions causing acute respiratory distress syndrome (ARDS). Severe COVID-19 patients are very breathless (and may be unable to breathe at a comfortable rate on slight moving around or even at rest) and breathe faster than usual, even when sitting still. They cannot finish a sentence without extra breaths. They may even avoid speaking. Their oxygen levels may have fallen so the urge to breathe faster is strong. Physicians usually measure breathing rates when assessing this condition. Normal adults breathe at about 12-18 breaths per minute when they are not thinking about it. In pneumonia the rate rises, sometimes markedly.

The preserved lung compliance in COVID-19 patients suggests that lung epithelial response to SARS-CoV-2 is not the single component of the disease. Moreover, the hyperinflammatory state demonstrated in COVID-19-related ARDS patients, also suggests an endothelial activation consecutive to the inflammatory response.

COVID-19 appears to damage the vasculature of the lungs. Particularly, it may induce capillary endothelial cell/microvascular dysfunction which may cause individual cell necrosis. Histopathologic basis of COVID-19 severe disease cases has been analyzed and showed in all cases diffuse alveolar damages involving activation of megakaryocytes, with platelet aggregation and platelet-rich clot formation, in addition to fibrin deposition.

Typically, COVID-19 patients are detected by a consistent clinical history, epidemiological contact, and a positive SARS-CoV-2 test. Specifically, SARS-CoV-2 infection can be confirmed by positive detection of viral RNA in nasopharyngeal secretions using a specific PCR test.

Also, according to the Berlin 2012 ARDS diagnostic criteria, COVID-19 ARDS is diagnosed when a COVID-19 patient presents (1) acute hypoxemic respiratory failure, (2) within 1 week of worsening respiratory symptoms; (3) bilateral airspace disease on chest x-ray, computed tomography, or ultrasound that is not fully explained by effusions, lobar or lung collapse, or nodules; and (4) cardiac failure is not the primary cause of acute hypoxemic respiratory failure. It also found that shortness of breath—also known as dyspnoea—is the only symptom of COVID-19 that is significantly associated with severe cases and with patients requiring admission to ICU. COVID-19 ARDS follows a predictable time course over days, with median time to intubation of 8.5 days after symptom onset. It is therefore important to monitor patients for the development of ARDS as their COVID-19 progresses. The respiratory rate and SpO₂ are two important parameters for judging patients' clinical condition and allowing early recognition of ARDS. A patient who fits any one of the following conditions may have severe disease and requires further evaluation/Respiratory rate ≥30 breaths/min; SpO₂≤92%; PaO₂/FiO₂≤300 mmHg

According to the present invention, methods of treating and compositions for use in said method may comprise further administering an anti-IL6 monoclonal antibody or an anti-IL6 receptor monoclonal antibody. By way of anti-IL6 monoclonal antibody or an anti-IL6 receptor monoclonal antibody which can be administered to infected patients, preferably COVID-19 patients, in combination with at least antagonist or inhibitor of chemokine receptor CXCR4, we may cite siltuximab (an anti-IL6 chimeric monoclonal antibody for treating Castleman's disease, and marketed under the tradename Sylvant®), olokizumab (an anti-IL6 humanized monoclonal antibody under development by the company R-Pharm for treating rheumatoid arthritis and also called CDP6038 or OKZ), sirukumab (an anti-iL6 human monoclonal antibody under development by the company Centocor for treating rheumatoid arthritis and also called CNTO0136), tocilizumab (an anti-IL6 human monoclonal antibody marketed by Genentech under the tradename Actemra® for treating rheumatoid arthritis) elsilimomab (an anti-human IL6 therapeutic antibody currently developed by Immunogen), clazakizumab (an anti-IL6 developed by BMS for treating rheumatoid arthritis, previously called BMS-945429 or ALD518), levilimab (BCD-089), CPSI-2364,UX-0061.

Methods of treating and compositions for use in a method according to the present invention, may further comprise administering a pharmaceutically acceptable vehicle, antiviral drugs, antibacterial drugs, antibiotics, PPI Inhibitors, quinoline compounds, zinc compounds, gamma globulin, hematopoietic cells, mesenchymal cells, anti-inflammatory drugs, vaccines, small interfering RNAs and microRNAs, immunomodulators, or plasma from convalescent patients. Preferably, said methods and compositions for use according to the present invention do not comprise further administration of any hydroxychloroquine or chloroquine to said COVID-19 patients.

Anti-viral drugs may be selected from the following drugs: nucleotide analogues, nucleoside analogues, nucleoside reverse transcriptase inhibitors (NRTIs), non-nucleoside reverse transcriptase inhibitors (NNRTIs), neuraminidase inhibitors, endonuclease inhibitors, adamantanes, protease inhibitors (PIs), integrase inhibitors (INSTIs), fusion inhibitors (FIs), chemokine receptor antagonists (CCRS antagonists), or siRNA.

By way of examples of anti-viral drug which may be administered to a COVID-19 patient in combination with the antagonist or inhibitor of chemokine receptor CXCR4 as described above, we may cite abacavir, acyclovir, adefovir, amantadine, ampligen, amprenavir (Agenerase™), arbidol, atazanavir, atripla, balavir, baloxavir marboxil (Xofluza™), biktarvy, boceprevir (Victrelis™), cidofovir, cohicistat (Tybost™), combivir, daclatasvir (Daklinza™), darunavir, delavirdine, descovy, didanosine, docosanol, dolutegravir, doravirine (Pifeltro™), ecoliever, edoxudine, efavirenz, elvitegravir, emtricitabine, enfuvirtide, entecavir, etravirine (Intelence™), famciclovir, fomivirsen, fosamprenavir, foscarnet, fosfonet, favipiravir, triazavirin, ganciclovir (Cytovene™), ibacitabine, ibalizumab (Trogarzo™), idoxuridine, imiquimod, imunovir, indinavir, inosine, interferon type I, interferon type II, interferon type III, lamivudine, letermovir (Prevymis™), lopinavir, loviride, maraviroc, methisazone, moroxydine, nelfinavir, nevirapine, nexavir, nitazoxanide, norvir, oseltamivir (Tamiflu™), PEG interferon α-2a, peginterferon α-2b, penciclovir, peramivir (Rapivab™), pleconaril, podophyllotoxin, pyramidine, raitegravir, remdesivir, ribavirin, rilpivirine (Edurant™), rimantadine, ritonavir, saquinavir, simeprevir (Olysio™), sofosbuvir, stavudine, telaprevir, telbivudine (Tyzeka™) tenofovir alafenamide, tenofovir disoproxil, tenofovir, tipranavir, trifluridine, trizivir, tromantadine, truvada, valaciclovir (Valtrex™), valgartciclovir, vicriviroc, vidarabine, viraniidine, zalcitabine, zanamivir (Relenza™), zidovudine.

Compositions and methods as described above may be administered via parenteral, oral, nasal, ocular, transmucosal, or transdermal. Most preferred routes of administration are intravenous, intramuscular or subcutaneous routes.

The specific therapeutic dose to be administered to a COVID-19 patients is the dose required to obtain therapeutic and/or prophylactic effects. This dose may be determined by the physician depending on the conditions of the patients, weight, age and sex, compound administered, the route of administration, etc. . . . . The dosage may be by a single dose, divided daily dose, or multiple daily doses. Alternatively, continuous dosing, such as for example, via a controlled (e.g., slow) release dosage form can be administered on a daily basis or for more than one day at a time.

According to the preferred embodiment as described above, plerixafor may be selected as antagonist or inhibitor of chemokine receptor CXCR4. It is then administered via the subcutaneous route at a dosage of around 10 to 40 μg/kg bid (20 to 80 μg/kg/day) and continuous intra-venous route at a dosage of around 10 to 120 μg/kg/hour. According to a most preferred embodiment, plerixafor is administered to COVID patients via intra-venous perfusion route a dosage of 30 μg/kg/day.

According to the present invention, methods and compositions are particularly useful for treating most vulnerable patients that have health conditions or comorbidities, such as hypertension, obesity, and/or diabetes. However, patients having history of myocardial infarction as well as cancer patients are excluded and thus are not treated with the compositions and methods according to the present invention. In addition, compositions and methods as described above are expected to have a protective effect on the heart, since Applicants have showed that administration for example of plerixafor improves cardiac remodeling quantified by Fulton index.

Said patients include patients selected from the group consisting of an end stage renal disease (ESRD) patient, a patient having chronic obstructive pulmonary disease (COPD), an AIDS patient, a diabetic patient, a patient subject to obesity, a patient subject to hypertension, a neonate, a transplant patient, a patient on immunosuppression therapy, a patient with malfunctioning immune system, an autoimmune disease patient, an elderly person in an extended care facility, a patient with autoimmune disease on immunosuppressive therapy, a burn patient, and a patient in an acute care setting.

CXCR4 antagonists suitable for use in accordance with the present invention can be administered alone but, in human therapy, will generally be administered in admixture with a suitable pharmaceutically acceptable vehicle, excipient, diluent, or carrier selected with regard to the intended route of administration and standard pharmaceutical practice. Such pharmaceutically acceptable vehicle or excipient may be present in an amount between 0.1% arid less than 100% by weight. Optimizing drug-excipient ratios are with the reach of a person with ordinary skill in art for instance the desired weight ratio of drug/excipient in the composition could be less than or equal to the ratio of solubilities of drug/excipient, in a suitable medium.

Pharmaceutical compositions of the present invention may be administered parenterally, for example, intravenously, intraarterially, intraperitoneally, intrathecally, intraventricularly, intrasternally, intracranially, intramuscularly, subcutaneously, or they may be administered by infusion or needle-free techniques.

For such parenteral administration they are best used in the form of a sterile aqueous solution, which may contain other substances, for example, enough salts or glucose to make the solution isotonic with blood. The aqueous solutions should be suitably buffered (preferably, to a pH of from about 3 to 9), if necessary. The preparation of suitable parenteral formulations under sterile conditions is readily accomplished by standard pharmaceutical techniques well known to those skilled in the art.

According to another embodiment of the present invention is directed to a composition for use in a method of treating and a method of treating COVID-19 disease in a subject in need thereof comprising: i) determining a viral load in a sample obtained from the subject by RT-PCR or other equivalent techniques; ii) comparing the viral load determined at step i) with a predetermined reference value; and iii) administering to the subject a therapeutically effective amount of at least one antagonist or inhibitor of chemokine receptor CXCR4 as described above. According to this embodiment, the sample is preferably a blood sample or a mucus sample.

Throughout this application, various references are referred to and disclosures of these publications in their entireties are hereby incorporated by reference into this application to more fully describe the state of the art to which this invention pertains.

### EXAMPLES

### Example 1: Effect of CXCR4 Antagonist on Lung Function

Mice which presented lung injury induced by cigarette smoke exposure with poly-IC instillations received daily subcutaneous injections of either PBS or 1 mg/kg plerixafor during the 5 weeks. Lung function was then assessed by measured by the FEV0.05/FVC ratio. FIG. 1 showed a statistically significant improvement of the murine lung function after treatment with 1 mg/kg plerixafor injection versus to PBS injection.

### Example 2: Effect of CXCR4 Antagonist on Cardiac Function

As in Example 1, mice which presented lung injury induced by cigarette smoke exposure with poly-IC instillations received daily subcutaneous injections of either PBS injection or 1 mg/kg plerixafor during the 5 weeks. Cardiac function and remodeling were then quantified by Fulton index, calculated as RV/(LV+S), with LV: left ventricle, RV: right ventricle, S: septum. FIG. 2 showed a statistically significant improvement of the murine cardiac function after treatment with 1 mg/kg plerixafor injection versus to PBS injection.

### Example 3: Effect of CXCR4 Antagonist on K18-hACE2 Mice Infected with SARS-CoV-2

Two groups of transgenic C57BL/6 mice expressing the human ACE2 receptor (K18-hACE2 mice) were allocated for this study. Infection with 5×10² TCID₅₀ of a clinical SARS-CoV-2 isolate SARS-CoV-2 was performed on 15 mice on day 0 by intranasal (IN) administration. The same day, infected mice were treated twice daily until termination with water for injection (group 1M) for 6 mice or Plerixafor at 10 mg/kg/day (group 2M) for 9 mice. During the study, mortality and morbidity observation and body weight measurements were performed. Mice were euthanized for ethical reasons when they achieved greater than 20% body weight loss.

### Example 3.1: Body Weight Loss

Body weight of mice was followed every day until termination.

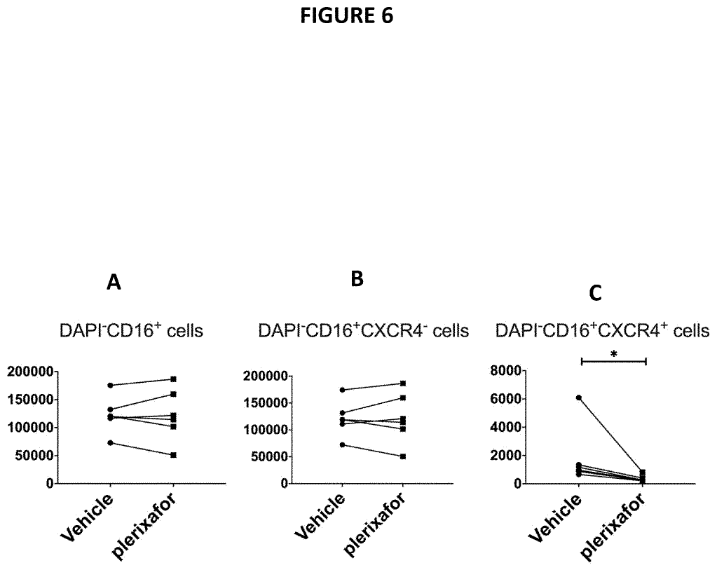

As shown in FIG. 6A, mice treated with Plerixafor demonstrated a significant reduction of body weight loss kinetic in comparison to vehicle mice (p<0.0011). Indeed, at day 5, Plerixafor treated mice lost in average 17.6%±1.28% of their body weight while vehicle treated mice lost in average 20.43%±2.88% of their body weight.

### Example 3.2: Mice Survival

Mice reaching a body weight loss superior to 20% were euthanized and the time of death was precisely recorded.

As shown in FIG. 6B, all mice from the vehicle group were euthanized on day 5 in the morning. Plerixafor treated mice were euthanized on day 5 (2 mice in the morning and 2 mice in the afternoon) and on day 6 (4 mice in the morning). Significant difference in time of death was observed between vehicle and plerixafor treated mice (Wilcoxon test p<0.05).

### Example 4: Clinical Trial 1 “LEONARDO”

pLErixafOr iN Acute Respiratory Distress Syndrome Related to cOvid-19 (Phase IIb)

### Example 4.1: Design

Randomized, double blind, placebo-controlled, multicenter trial with parallel groups.

### Example 4.2: Primary Objective:

To demonstrate that Plerixafor is able to decrease the rate of invasive mechanical ventilation in severe COVID-19 patients admitted in Intensive Care Unit (ICU).

### Example 4.3: Primary Outcome

Percentage of patients alive and who did not require invasive mechanical ventilation (composite primary endpoint including mortality and or time to require invasive mechanical ventilation, whichever occurs first) from D1 to D28.

### Example 4.4: Secondary Outcomes

To evaluate the efficacy of Plerixafor compared to placebo on
1. Mortality [Time Frame: D1-D90]
2. Ventilation-free days [Time Frame: D1-D28]
3. Duration of ventilation [Time Frame: D1-D90]
4. Duration of ICU stay [Time Frame: D1-D90]
5. Respiratory function including Forced Expiratory Volume in one sec (FEV1), Forced Vital Capacity (FVC), Partial arterial pressure in Oxygen (PaO₂) and Transfer Lung Capacity for carbon monoxide (TLCO) [Time Frame: D90]
6. Clinical improvement [Time Frame: D1, D8, D14, D28, D90]
7. CRP, fibrinogen and D-dimers levels [Time Frame: D1, D3, D8, D14, D28]
8. To evaluate the safety and tolerability of Plerixafor compared to placebo on Safety (AEs, cardiac monitoring, vital signs, lab tests)

### Example 4.5: Exploratory Outcomes

To assess the effect of Plerixafor on
1. Blood CXCR4-positive neutrophil count, CXCR4-positive fibrocyte count, CXCL12, IL-6, IL-8, TNFa concentrations [Time Frame: D1, D3, D8, D28]
2. Blood CD34+ cell count [Time Frame: D1, D3, D8, D28]
3. Blood NETosis [Time Frame: D1, D3, D8, D28]
4. Blood CD4+ cells, CD8+ cells, regulatory T cells, NK cells. [Time Frame: D1, D3, D8, D28]

### Example 4.6: Inclusion Criteria

Male or female ≥18 years of age, using contraceptive consistent with local regulations regarding the methods of contraception for those participating in clinical studies (see section 10.4),
Willing and able to provide written informed consent (or provided by legally acceptable representative if he/she is present),
Recently admitted in ICU (within 48 hours) for COVID-19 related respiratory failure. ICU or equivalent medical structure according to country specificities e.g., Acute Respiratory Care Unit, High Dependency Care Unit if they can provide:
   continuous IV infusion,
   continuous cardiotelemetry, respiratory rate, percutaneous oxygen saturation monitoring
   high flow nasal oxygen
Not requiring immediate (within 24-36 hours) invasive mechanical ventilation according to investigator judgment,
Confirmed pneumoniae due to SARS-CoV-2 with
   Laboratory-confirmed SARS-CoV-2 infection as determined by RT-PCR (in nasopharynx or throat samples) or other commercial or public health assay in any specimen, performed within 2 weeks prior to randomization,
Acute respiratory failure requiring oxygen support (≥5 L/min) to achieve a transcutaneous oxygen saturation >94%,
Estimated glomerular filtration rate (eGFR) >50 mL/min/1.73m² by the CKD-EPI (Chronic Kidney Disease-Epidemiology Collaboration) equation. (Calculation is provided in Section 10.8).

COVID-19 Vaccinated patients can be also included in the study.

### Example 4.7: Exclusion Criteria

Pregnancy or breast feeding,
Do-not-intubate order at admission,
Anticipated transfer to another hospital, which is not a study site within 72 hours,
Need for Invasive mechanical ventilation at time of inclusion,
Evidence of bacterial pneumopathy or active infection other than SARS-Cov-2 (laboratory confirmation),
Primitive pulmonary arterial hypertension,
Cardio-vascular co-morbidity:
   History of vascular ischemic events (myocardial infarction or stroke) or congestive heart failure or peripheral arterial disease,
   History or current significant cardiac rhythm disorders (e.g., ventricular tachycardia),
   Known medical history of proven symptomatic postural hypotension,
History of evolutive cancer including acute and chronic leukaemia,
Inadequate haematological function defined by:
   Absolute neutrophil count (ANC) <1.0×10⁹/L,
   Haemoglobin <9.0 g/dL (90 g/L),
   Platelets <100×10⁹/L,
Kaliemia <3.5 mmol/L and/or Calcemia <2.2 mmol/L,
Inadequate hepatic function defined by Aspartate aminotransferase (AST) and/or Alanine Aminotransferase (ALT) >3× upper limit of normal (ULN) and/or Total bilirubin >2× ULN
Patients with known allergy to Plerixafor or its excipients.

### Example 4.8: Dosage Schedule

Plerixafor 30 μg/kg/hour continuous intravenous infusion for 7 days or Placebo similar modalities for infusion

### Example 4.9: Number of Patients to be Included

Sample size is based on the following assumptions:
1. Current estimate of the proportion of patients in ICU with critical stage COVID-19 and requiring mechanical ventilation (50% as per Bichat and Bordeaux CHUs),
2. Estimate that Plerixafor will reduce the proportion of invasive mechanical ventilation in patients with severe COVID-19 from 50% to 27% (i.e., a 46% decrease versus placebo),
3. A two-sided analysis, with α=0.05.

With the above-mentioned assumptions, 100 patients in Plerixafor group and 50 in placebo group will be required to detect a significant decrease in the proportion of patients requiring invasive mechanical ventilation with a power of 80%.

### Example 5: Clinical Trial 1 “LEONARDO-1”

pLErixafOr iN Acute Respiratory Distress syndrome related to cOvid-19 (Phase IIb)

### Example 5.1: Design

Randomized, double blind, placebo-controlled, multicenter trial with parallel groups.

### Example 5.2: Primary Objective

To demonstrate that the CXCR4 antagonist plerixafor on top of the best available treatment is able to increase Ventilator-free days in COVID-19-related ARDS patients.

### Example 5.3: Primary Outcome

Ventilator-free days (VFDs) at 28 days are one of several organ failure-free outcome measures to quantify the efficacy of therapies and interventions. VFDs are typically defined as follows:
a. VFDs=0 if subject dies within 28 days of mechanical ventilation.
b. VFDs=28−x if successfully liberated from ventilation x days after initiation.
c. VFDs=0 if the subject is mechanically ventilated for >28 days.

### Example 5.4: Secondary Outcomes

1. Day-28 mortality [Time Frame: 28 days after randomization]Mortality rate evaluated 28 days after randomization
2. Day-90 mortality [Time Frame: 90 days after randomization], Mortality rate evaluated 90 days after randomization
3. Percentage of patients free of invasive ventilation within 28 days [Time Frame: At Day28]
4. Number of ICU-free days within 28 days [Time Frame: at Day28]
5. CT-scan presence of lung fibrosis at month 6 [Time Frame: at Month 6]

16. Safety at days 1, 7, 14, 28 (pulmonary embolism, myocardial infarction, cardiac arrhythmia, arterial hypotension, anemia, thrombocytopenia, hyperleukocytosis, estimated GFR, proteinuria)
7. Blood levels of CRP, ferritin, D-dimers at Day 1, Day 3 and Day 7 and Day 28

### Example 5.5: Other Secondary Outcomes in a Sub-Study

1. Blood neutrophils and fibrocytes count at Day 1, Day 3 and Day 7
2. Blood CXCR4-positive neutrophils, CXCR4-positive fibrocytes, CD34⁺ cells count at Day 1, Day 3 and Day 7
3. Blood CXCL12 concentrations at Day 1, Day 3 and Day 7
4. Blood NETosis at Day 1, Day 3 and Day 7
   a. MPO and H3 citrullinated
   b. DNase

### Example 5.6: Inclusion Criteria

Male or female ≥18 years of age
Willing and able to provide written informed consent (or provided by a proxy)
Currently hospitalized with laboratory confirmed COVID-19 infection
Requiring invasive mechanical ventilation of less than 48 h
Standard of care recommended by FDA-EMA at the beginning of the trial
With diagnosis of ARDS according to Berlin criteria

### Example 5.7: Exclusion Criteria

Patient with a do-not-resuscitate order (DNR order)
Pregnancy or breast feeding
Known bacterial pneumonia
Current myocardial infarction
Known cancer (solid or blood) during the last 5 previous years
Primitive pulmonary hypertension, lung fibrosis
Hypersensitivity to Plerixafor
Neutrophil concentration >50 000/mm³ at any time

### Example 4.8: Dosage Schedule

Plerixafor subcutaneously 10 to 40 μg/kg bid (20 to 80 μg/kg/day) for 14 days.

In HIV clinical trials, plerixafor (AMD3100) was administered for 10 days by continuous intravenous infusion in an open-label dose escalation study from 2.5 to 160 μg/kg/h.

In WHIM syndrome, plerixafor was administered for 6 months by subcutaneously injection (10 to 20 μg/kg bid for 6 months).

Dose must be adjusted when creatinine clearance <50 ml/min

### Example 5.9: Number of Patients to be Included

Based on recently submitted study, ARDS patients treated by Tocilizumab had: VFDs=14.86+/−5.36 at days 28.

The hypothesis is that plerixafor treatment in combination with best available treatment as described in herein above, will increase VFDs by 2 days. 4 groups of patients (placebo, pleri 10, pleri 20, pleri 40 μg/kg bid) will be planned. To get a power of 90% with an alpha risk of 5%, we have to include 132 patients per arm.

### Example 6: Clinical Trial 2 “PLANET”

PlerixAfor iN modEraTe forms of COVID-19 (Phase IIb)

### Example 6.1: Design

Randomized, double blind, placebo-controlled, multicenter trial with parallel groups.

### Example 6.2: Primary Objective

The choice between choices 1 and 2 will depend on the percentage of patients moving from moderate to severe forms of COVID-19 (data coming soon, DISCOVERY/CORIMMUNO trials)

Choice 1:

To demonstrate that the CXCR4 antagonist Plerixafor on top of the best available treatment can decrease the time to reach a mild form of COVID-19.

Choice 2:

To demonstrate that the CXCR4 antagonist Plerixafor on top of the best available treatment is able to decrease the percentage of patients with moderate forms of COVID-19 that are either deceased or need ventilation (mechanical or noninvasive) at day 14.

### Example 6.3: Primary Outcome

Choice 1:

Time to reach mild form of COVID-19, defined by no need of ventilatory support (WHO criteria).

Choice 2:

Percentage of patients that need ventilation (mechanical or noninvasive) or deceased at day 14 Example 6.4: Secondary outcomes:
1. Percentage of patients that have a mild form of COVID-19 at day 14
2. Difference between initial CT-scan (diagnosis) and 3-month CT-scan (after randomization) [Time Frame: at Month 3]
3. Difference between initial TDI and final TDI [Time Frame: at Day 14]
4. 6 min walk test distance (m) performed at ambient air at day 14]
5. Percentage of patients with SpO2<90% during 6 min walk test performed at ambient air
6. Day-28 mortality [Time Frame: 28 days after randomization]Mortality rate evaluated 28 days after randomization
7. Number of ICU-free days within 28 days [ Time Frame: at Day 28]
8. Maximal oxygen rate within 28 days [Time Frame: at Day 28]
9. Number of days alive outside hospital within 28 days [Time Frame: at Day 28]
10. Number of days alive with oxygen therapy within 28 days [Time Frame: at Day28
11. Safety at days 1, 7, 14, 28 (pulmonary embolism, myocardial infarction, cardiac arrhythmia, arterial hypotension, anemia, thrombocytopenia, hyperleukocytosis, estimated GFR, proteinuria)
12. Blood levels of CRP, ferritin, D-dimers at Day 1, Day 3 and Day 7 and Day 28 Example 6.5: Other secondary outcomes in a Sub-study:
13. Blood neutrophils and fibrocytes count at Day 1, Day 3 and Day 7
14. Blood CXCR4-positive neutrophils, CXCR4-positive fibrocytes, CD34⁺ cells count at Day 1, Day 3 and Day 7
15. Blood CXCL12 concentrations at Day 1, Day 3 and Day 7
16. Blood NETosis at Day 1, Day 3 and Day 7
   c. MPO and H3 citrullinated
   d. DNase

### Example 6.6: Inclusion Criteria

Male or female ≥18 years of age
Willing and able to provide written informed consent (or provided by a proxy)
Currently hospitalized with laboratory confirmed COVID-19 infection
With moderate form of COVID-19, defined by the need for oxygen therapy between 3 and 5 L/min to obtain a percutaneous oxygen saturation greater than 97% and a respiratory rate <25 breaths/min without the need for invasive ventilation.
Standard of care recommended by FDA-EMA at the beginning of the trial

### Example 6.7: Exclusion Criteria

Patients hospitalized in ICU
Patients requiring invasive mechanical ventilation
Pregnancy or breast feeding
Known bacterial pneumonia
Current myocardial infarction
Current pulmonary embolism
Known cancer (solid or blood) during the last 5 previous years
Primitive pulmonary hypertension, lung fibrosis
Hypersensitivity to Plerixafor
Neutrophil concentration >50 000/mm³ at any time

### Example 6.8: Dosage Schedule

Plerixafor subcutaneously 10 to 40 μg/kg bid (20 to 80 μg/kg/day) for 14 days In HIV clinical trials, plerixafor (AMD3100) was administered for 10 days by continuous intravenous infusion in an open-label dose escalation study from 2.5 to 160 μg/kg/h.

In WHIM syndrome, plerixafor was administered for 6 months by subcutaneously injection (10 to 20 μg/kg bid for 6 months).

Dose must be adjusted when creatinine clearance <50 ml/min

### Example 7: Plerixafor Inhibits RNA-Viral Infection-Induced NETosis

Five groups of C57B1/6 mice were allocated for this study. Infection with RNA virus was performed on 10 mice on day 0 by intranasal (IN) administration of PBS containing 50 plaque forming units (PFU).

2 mice were treated intranasally with 50 μl of PBS (group 1M-non infected).

On day 1, infected mice were treated twice daily with water for injection (group 2M and 4M) or Plerixafor at 3 mg/kg/day (group 3M and 5M). During the study, mortality and morbidity observation and body weight measurements were performed.

At day 7, mice from group 1M, 2M and 3M were euthanized, and blood was collected for NETosis analysis in plasma (citrullinated histone H3 and DNA).

The study was conducted according to Table 1 for study design and Table 2 for study timeline.

TABLE 1
Study design

| | | IAV | | | | | |
|---|---|---|---|---|---|---|---|
| | | (Intranasal | | | | | |
| | | injection | | | | | |
| | | of 50 μl - | | Dose | Dose | | Dose |
| Group | N= | 50 PFU) | Treatment | level | volume | ROA | regimen |
| 1M | 2 | PBS | — | — | — | — | — |
| 2M | 5 | ✓ | Vehicle | — | 100 | SC | Twice |
| | | | (water for | | μl | | a day |
| | | | injection) | | | | |
| 3M | 5 | ✓ | Plerixafor | 1.5 | | | |
| | | | | mg/kg | | | |
| 4M | 4 | ✓ | Vehicle | — | | | |
| | | | (water for | | | | |
| | | | injection) | | | | |
| 5M | 4 | ✓ | Plerixafor | 1.5 | | | |
| | | | | mg/kg | | | |

TABLE 2
Study timeline

| Study | | |
|---|---|---|
| Day/week* | Procedure | Remarks |
| D 0 | IAV infection, 50 | Except for group 1M |
| | PFU in 50 μl, IN | (Non-infected), 50 μl |
| | | injectable PBS, IN |
| D 1 to | treatment every day | SC injection twice |
| D 6 | (Vehicle, Plerixafor) | a day, 100 μl |
| D 7 | termination for group | Blood collection |
| | 1M, 2M, 3M | |

On day 7, mice from groups 1M, 2M and 3M were euthanized. Blood was collected by cardiac puncture after opening the thoracic cavity.

### Example 7.1: Blood Samples Preparation

Blood samples were centrifuged for 15 minutes at 1500 g at 4° C. for plasma separation. Plasma was harvested and transferred immediately to clean 1.5 ml Eppendorf tubes and stored at −70° C. or in dry ice for shipment.

### Example 7.2: NETosis Analysis in Plasma

Plasma samples were sent to external laboratory at Bordeaux Hospital U1034 unit for NETosis analysis. NETosis was evaluated by dosing the citrullinated histone H3 and DNA in plasma. For citrullinated histone H3 dosing, the technique is adapted from a method described by Thalin et al. (Validation of an enzyme-linked immunosorbent assay for the quantification of citrullinated histone H3 as a marker for neutrophil cellular traps in human plasma. Immunol Res. 2017; 65: 706-712) with a partial used of the Cell death detection kit (Roche).

Wells were coated with an anti-histone H11-4 antibody overnight at +4° C. After 3 washes with washing buffer, 40 μl of plasma+60 μl of incubation buffer were incubated for 90 minutes under agitation. After 3 washes, 100 μl of Rabbit polyclonal to Histone H3 (citrulline R2+R8+R17) antibody diluted 1:2000 in incubation buffer were applied to each well for 90 minutes at room temperature under agitation. After 3 washes, 100 μl of goat anti-rabbit HRP antibody diluted 1:6000 in incubation buffer were added. Plate was incubated 60 minutes at room temperature under agitation. After 3 washes, 100 μl of TMB were added for 5-10 minutes and the reaction was stopped with 100 μl of H2SO4 0.25M.

The absorbance was read at 450nm (reference 620 nm).

Blood was collected for mice of group 1M, 2M and 3M at termination on day 7 (5 mice per group for groups 2M and 3M and 2 mice for group 1M). Plasma samples were harvested and NETosis parameters were evaluated by dosing the citrullinated histone H3 and the plasmatic DNA concentration.

### Example 7.3: Evaluation of the Level of Citrullinated Histone H3

The level of Citrullinated histone H3, a biomarker of neutrophil cellular trap, was evaluated in plasma. Individual and group average citrullinated histone H3 levels are presented in Table 3 and FIG. 3.

Citrullinated histone H3 level non-infected mice plasma was detected at low concentration. The infection with RNA virus increased the level of this marker (2.124±0.563 for RNA virus infected group vs 0.113±0.008 for non-infected group). Plerixafor treatment at 3 mg/kg/day slightly decreased the level of citrullinated histone H3 (1.428±0.799 for RNA virus infected+Plerixafor 3 mg/kg/day vs 2.124±0.563 for RNA virus infected group).

TABLE 3
Evaluation of citrullinated histone H3
in plasma (in absorbance at 450 nm)

| | Mean Citrullinated | |
|---|---|---|
| | Histone H3 | SD |
| Group 1M - Non infected | 0.113 | 0.008 |
| Group 2M - RNA virus infected | 2.124 | 0.563 |
| Group 3M - RNA virus infected + | 1.428 | 0.799 |
| Plerixafor 3 mg/kg/day | | |

### Example 8: Plerixafor Inhibits COVID-19 Patients' Sera Induced Neutrophils' Migration

Neutrophils were isolated from 6 patients of the COBRA cohort (COPD and asthma patient cohort). Neutrophils 5×10⁵ were pre-treated with plerixafor (25 μg/mL) for 15 min at 37° C. or with HBSS vehicle. Cells were next plated in the upper compartment of a modified Boyden chamber for migration assay in 0.2 mL HBSS, containing 0.5% HSA medium. HBSS, 0.5% HSA or HBSS, 0.5% HSA+sera (10% diluted) from severe COVID-19 patients were added to the bottom compartment of each well. The migration assay lasted 3hours, after which cells in the lower compartment were collected and stained with DAPI, anti-CD16 and anti-CXCR4 antibodies. Cell identities and counts were analysed by FACS method.

### Example 8.1: Isolation of Human Neutrophils from Whole Blood

Neutrophils are isolated according to the method described by Hard, et al., (Infiltrated neutrophils acquire novel chemokine receptor expression and chemokine responsiveness in chronic inflammatory lung diseases. J. Immunol. Baltim. Md 1950. 181:8053-8067). Neutrophils were freshly isolated from blood of COPD patients over isotonic Percoll density gradient. The neutrophils were recovered, washed twice with HBSS containing 20 mM HEPES and 0.1% (w/v) BSA and resuspended in HBSS, 20 mM HEPES, and 0.1% (w/v) BSA at a cell concentration of 5×10⁵ in 0.2 mL.

### Example 8.2: Effect of COVID-19 Serum on Neutrophils' Migration

Neutrophil migration was assessed using a Boyden chamber assay, using transwell inserts (pore size 8 um). A total of 5x 10⁵ neutrophils cells in 0.2 mL HBSS, containing 0.5% HSA were added to the upper compartment of each well. When indicated, neutrophils were pretreated for 15 min at 37° C. with plerixafor an antagonist of CXCR4 (25 μg/mL). HBSS, 0.5% HSA and Sera (10% diluted) from severe COVID-19 patients was added to the bottom compartment of each well. After 3 h, the content of bottom compartment was washed with 1 mL and lower chamber cell content was retrieved in a FACS tube (1 inferior chamber cell content per FACS tube) and process for DAPI staining was performed to exclude dying cells.

After neutrophils' recovery from lower chamber, FACS tubes were centrifuged 5 min, 4° C. at 500 g. Fixation and permeabilization of cells were as follow: 0.3 mL of Cytofix/Cytoperm were added for 15 min (tube are protected from light). Next, 400 μL of permeabilization buffer was added and cells were processed for centrifugation for 5 min, 4° C., 400 g. Supernatant was discarded, and cells were processed for antibody staining. Cells were incubated 40 min at 4° C. with anti-CD16 to account for all neutrophils, anti-CXCR4 to specifically account for CXCR4 expressing neutrophils. Next, 400 μL of permeabilization buffer was added, and cells were centrifuged 5 min at 4° C., 400 g. After supernatant was discarded, cells were incubated with appropriate secondary antibody for 40 min at 4° C. Finally, cells were incubated with DAPI at 4° C. After DAPI incubation (to exclude dying cells from FACS counts), 400 μL of PBS 1× were added and cells were centrifuged for 5 min at 4° C., 400 g. Cells were resuspended in 300 μL of PBS 1×. To obtain absolute values of migratory cells, flow cytometric counts for each condition were obtained during a constant predetermined time period (1 min).

TABLE 4
Study design

| Condition | Cells | Pre-Treatment | Upper chamber | Lower chamber | Assay |
|---|---|---|---|---|---|
| 1 | Neutrophils | Plerixafor | HBSS + | HBSS + 0.5% HSA + | migration |
| | | (15 min) | 0.5% HSA | No serum | assay 3 |
| 2 | | Vehicle | HBSS + | HBSS + 0.5% HSA + | hours + |
| | | (15 min) | 0.5% HSA | No serum | FACS |
| 3 | | Plerixafor | HBSS + | HBSS + 0.5% HSA + | |
| | | (15 min) | 0.5% HSA | 10% COVID-19 Sera | |
| 4 | | Vehicle | HBSS + | HBSS + 0.5% HSA + | |
| | | (15 min) | 0.5% HSA | 10% COVID-19 Sera | |

Each condition treatment was run in duplicate.

The effect of COVID-19 sera from patients was evaluated and showed in FIG. 4.

Neutrophil migration from patients with stable COPD (n=6) in response to 10% sera from with severe COVID in the presence of 25 μg/mL plerixafor (red circle symbols) or vehicle (black circle symbols). Comparison of DAPI⁻CD16⁺ (FIG. 4A), DAPI⁻CD16⁺ CXCR4⁻ (FIG. 4B) and DAPI⁻CD16⁺ CXCR4⁺ (FIG. 4C) cell migration in response to sera from patients with severe COVID. Results were expressed with symbols indicating individual subject values. * P<0.5, Wilcoxon matched pair test.

FACS analyses established that the fraction of CXCR4+ neutrophils among total DAPI-CD1630 is around 1% (Table 5) in presence of 10% serum. Which is in keeping with the fact that in non-inflammatory or non-COVID conditions immature CXCR4+ neutrophils are only present in the bone marrow but have minimal release in the blood stream.

TABLE 5
Percentage of DAPI−CD16+CXCR4+ neutrophils
among DAPI−CD16+ neutrophils

| | DAPI−CD16+CXCR4+ cells | | | | | |
|---|---|---|---|---|---|---|
| | Sg87 | Sg88 | Sg89 | Sg92 | Sg95 | Sg3 |
| Percentage of | 1 | 1 | 1 | 1 | 1 | 5 |
| DAPI−CD16+CXCR4+ | | | | | | |
| neutrophils' among | | | | | | |
| DAPI−CD16+ | | | | | | |

The migratory response of DAPI-CD16+, DAPI-CD16+, CXCR4− and DAPI-CD16+CXCR4+ cells was evaluated. Severe COVID-19 serum induced a migratory response from neutrophils. Indeed, neutrophils migrate significantly more upon exposure to COVID-19 serum (P<0.05). CXCR4+ neutrophils migrate 2 times (Sg87) to 1345 times (Sg89) more in response to COVID-19 serum compared to 0% serum condition (Table 6). (raw data of FACS counts and separate raw data for each patient is provided in annex section).

TABLE 6
Ratio of DAPI−CD16+CXCR4+ neutrophils' migration increase
in response to 10% COVID-19 serum vs 0% serum condition

| | DAPI−CD16+CXCR4+ cells | | | | | |
|---|---|---|---|---|---|---|
| | Sg87 | Sg88 | Sg89 | Sg92 | Sg95 | Sg3 |
| Ratio of | 2.2 | 83.7 | 1344.5 | 3.2 | 7.7 | 221.7 |
| DAPI−CD16+CXCR4+ | | | | | | |
| neutrophils' migration | | | | | | |
| in response to10% | | | | | | |
| COVID19 serum vs | | | | | | |
| 0% serum | | | | | | |

### Example 8.3: Effect of Plerixafor on Neutrophils' Migration

Next, the effect of plerixafor (25 μg/mL) was evaluated in the three neutrophils population, namely DAPI-CD16+, DAPI-CD16+CXCR4− and DAPI-CD16+CXCR4+.

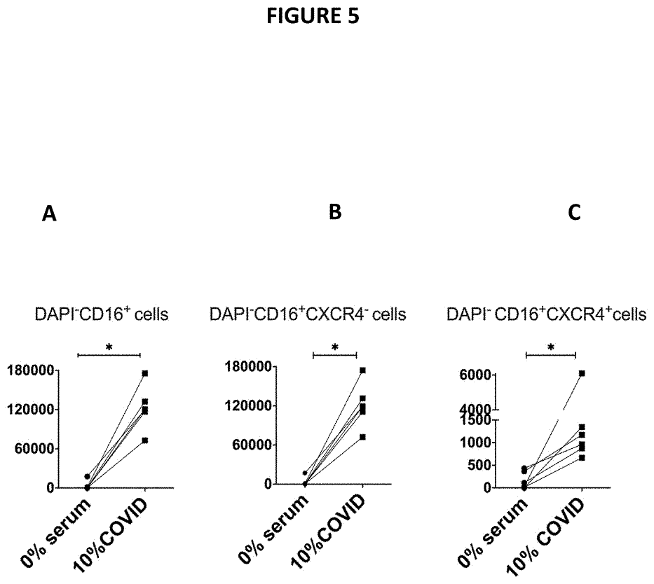

As showed in FIG. 5, neutrophil migration from patients with stable COPD (n=6) in response to 10% sera from with severe COVID in the presence of 25 μg/mL plerixafor (red circle symbols) or vehicle (black circle symbols). Comparison of DAPI⁻CD16⁺ (FIG. 5A), DAPI⁻CD16⁺ CXCR4⁻ (FIG. 5B) and DAPI−CD16+CXCR4+ (FIG. 5C) cell migration in response to sera from patients with severe COVID. Results are expressed with symbols indicating individual subject values. * P<0.5, Wilcoxon matched pair test.

The CXCR4 antagonist plerixafor did not significantly reduce serum-induced recruitment of neutrophils total population (FIG. 5A). Not surprisingly, plerixafor had no significant effect on the migration of CXCR4 negative neutrophils (FIG. 5B). This is in keeping with the small percentage that DAPI-CD16+CXCR4+ represent (around 1%). In contrast, plerixafor strongly and reproducibly decreased the migration of CXCR4 positive neutrophils (FIG. 5C). Plerixafor decreased CXCR4+ neutrophils by 75.8%±5.9% (Table 7).

TABLE 7
Percentage of DAPI−CD16+CXCR4+ neutrophils' migration decrease
in presence of plerixafor in response to COVID-19 serum

| | DAPI−CD16+CXCR4+ cells | | | | | |
|---|---|---|---|---|---|---|
| | Sg87 | Sg88 | Sg89 | Sg92 | Sg95 | Sg3 |
| % of | 72.9% | 75.5% | 70.0% | 77.6% | 72.4% | 86.6% |
| neutrophils' | | | | | | |
| migration | | | | | | |
| decrease with | | | | | | |
| plerixafor vs | | | | | | |
| vehicle in | | | | | | |
| response to | | | | | | |
| COVID-19 | | | | | | |
| serum | | | | | | |

1-15. (canceled)

16. A method of treating COVID-19 in a patient, comprising administering to the patient a therapeutically effective amount of at least one antagonist or inhibitor of chemokine receptor CXCR4, wherein said antagonist or inhibitor of chemokine receptor CXCR4 is distinct from hydroxychloroquine.

17. The method according to claim 16, wherein said antagonist or inhibitor of chemokine receptor CXCR4 is an indole-based compound, a N-substituted indole compound, a bicyclam compound, a cyclam mimetic compound, a para-xylyl-enediamine-based compound, a guanidine-based antagonist compound, a tetrahydroquinolines-based compound, or a 1,4-phenylenebis(methylene) compound.

18. The method according to claim 16, wherein said antagonist or inhibitor of chemokine receptor CXCR4 is chosen among plerixafor (AMD3100), burixafor (TG-0054), JM1657, AMD3329, AMD3465, AMD070, MSX-122, CTCE-9908, WZ811, or BKT-140.

19. The method according to claim 16, wherein said antagonist or inhibitor of chemokine receptor CXCR4 is administered via a route chosen among parenteral, oral, nasal, ocular, transmucosal, or transdermal.

20. The method according to claim 16, wherein said antagonist or inhibitor of chemokine receptor CXCR4 is administered via intravenous, intramuscular or subcutaneous route.

21. The method according to claim 16, wherein said antagonist or inhibitor of chemokine receptor CXCR4 is plerixafor and wherein plerixafor is administered via the subcutaneous route at a dosage of around 5 to 80 μg/kg bid (20 to 80 μg/kg/day) and continuous intravenous route at a dosage of around 10 to 120 μg/kg/hour.

22. The method according to claim 16, wherein the patient is in moderate to advanced stage of COVID-19 infection.

23. The method according to claim 16, comprising further treating comorbidity or multiple comorbidity of the patient.

24. The method according to claim 23, wherein patients with comorbidity or multiple comorbidity comprises obese patients, patients having hypertension, patients having chronic obstructive pulmonary disease (COPD), end stage renal disease (ESRD) patients, AIDS patients, diabetic patients, neonates, transplant patients, patients on immunosuppression therapy, patient with malfunctioning immune system, autoimmune disease patient, elderly patient in an extended care facility, patient with autoimmune disease on immunosuppressive therapy, burn patient, or patient in an acute care setting.

25. The method according to claim 16, further comprising administering a pharmaceutically acceptable vehicle.

26. The method according to claim 16, further comprising administering to said COVID-19 patient an anti-IL6 monoclonal antibody or anti-IL6 receptor monoclonal antibody, antiviral drugs, antibacterial drugs, antibiotics, PPI Inhibitors, quinoline compounds, zinc compounds, gamma globulin, hematopoietic cells, mesenchymal cells, anti-inflammatory drugs, vaccines, small interfering RNAs and microRNAs, immunomodulators, or convalescent plasma.

27. The method according to claim 26, wherein the anti-IL6 monoclonal antibody or anti-IL6 receptor monoclonal antibody is chosen among siltuximab, olokizumab, sirukumab, tocilizumab, elsilimomab, clazakizumab, levilimab, CPSI-2364, or ALX-0061.

28. The method according to claim 26, wherein antiviral drugs are chosen among abacavir, acyclovir, adefovir, amantadine, ampligen, amprenavir (Agenerase™) arbidol, atazanavir, atripla, balavir, baloxavir marboxil (Xofluza™), biktarvy, boceprevir (Victrelis™), cidofovir, cobicistat (Tybost™), combivir, daclatasvir (Daklinza™), darunavir, delavirdine, descovy, didanosine, docosanol, dolutegravir, doravirine (Pifeltro™), ecoliever, edoxudine, efavirenz, elvitegravir, emtricitabine, enfuvirtide, entecavir, etravirine (Intelence™), famciclovir, fomivirsen, fosamprenavir, foscarnet, fosfonet, favipiravir, triazavirin, ganciclovir (Cytovene™), ibacitabine, ibalizumab (Trogarzo™), idoxuridine, imiquimod, imunovir, indinavir, inosine, interferon type I, interferon type II, interferon type III, lamivudine, letermovir (Prevymis™), lopinavir, loviride, maraviroc, methisazone, moroxydine, nelfinavir, nevirapine, nexavir, nitazoxanide, norvir, oseltamivir (Tamiflu™), PEG interferon α-2a, peginterferon α-2b, penciclovir, peramivir (Rapivab™), pleconaril, podophyllotoxin, pyramidine, raltegravir, remdesivir, ribavirin, rilpivirine (Edurant™), rimantadine, ritonavir, saquinavir, simeprevir (Olysio™), sofosbuvir, stavudine, telaprevir, telbivudine (Tyzeka™), tenofovir alafenamide, tenofovir disoproxil, tenofovir, tipranavir, trifluridine, trizivir, tromantadine, truvada, valaciclovir (Valtrex™), valganciclovir, vicriviroc, vidarabine, viramidine, zalcitabine, zanamivir (Relenza™), zidovudine.

29. A method of treating COVID-19 in a patient in need thereof comprising:
i) determining a viral load in a sample obtained from a subject by RT-PCR or other equivalent techniques;
ii) comparing the viral load determined at step i) with a predetermined reference value; and
iii) administering to the subject a composition comprising a therapeutically effective amount of at least one antagonist or inhibitor of chemokine receptor CXCR4.

30. The method according to claim 29, wherein the sample is a blood sample or a mucus sample.
